(19) United States
(12) Patent Application Publication
(10) Pub. No.: US 2011/0071514 A1
(43) Pub. Date: Mar. 24, 2011

(54) METHOD AND SYSTEM FOR CONTROLLING RADIO FREQUENCY OUTPUT ACCORDING TO CHANGE IN IMPEDANCE OF BIOLOGICAL CELLS

(75) Inventors: Kyong-Min Shin, Seoul (KR); Hong-chea Jung, Gimpo-Si (KR); Young-jin Choi, Seoul (KR); Jun-hyok Lee, Kimpo-Si (KR); Jae-Bok Kim, Suwon-Si (KR); Guk-joo Na, Kimpo-Si (KR); Kye-joo Kim, Kimpo-Si (KR)

(73) Assignees: Taewoong Medical Co., Ltd, Kimpo -Si (KR); Kyong Min Shin, Seoul (KR)

(21) Appl. No.: 12/730,798

(22) Filed: Mar. 24, 2010

(30) Foreign Application Priority Data
Sep. 23, 2009 (KR) 10-2009-0090125

(51) Int. Cl. A61B 18/18

(52) U.S. Cl. 606/33

(57) ABSTRACT

Disclosed herein is a method and system for controlling radio frequency (RF) output according to the change in impedance of biological cells. In the method of controlling radio frequency (RF) output according to a change in impedance of biological cells, phases of RF voltage and current applied to the electrode are detected and a difference between the phases is obtained. A change in impedance such as a resistance component and a capacitive reactance component (XC) of the biological cells at the time of performing cauterization is obtained. An inductive reactance component (XL) is connected to an RF output terminal so as to cancel the capacitive reactance component (XC) (XC=XL), thus enabling the output impedance to have a resistance component.

### BACKGROUND OF THE INVENTION

1. Field of the Invention

The present invention relates, in general, to a method and system for controlling radio frequency (RF) output according to the change in impedance of biological cells. More particularly, the present invention relates to a method and system for controlling RF output according to the change in impedance of biological cells, which classifies the change in impedance of biological cells into a change in a resistance component and a change in a reactance component at the time of performing cauterization, so that the capacitive reactance of the biological cells is canceled by connecting an inductive reactance component to an output terminal and only a pure resistance component remains, thus improving the efficiency of the cauterization.

2. Description of the Related Art

Generally, as methods of treating cancerous tissue generated in the bodily organs of a human being, for example, the liver, there are methods of suppressing the growth of cancerous tissue and eliminating the cancerous tissue using drugs and radiotherapy without performing surgery, and methods of surgically eliminating cancerous tissue by performing surgery.

Of the above-described treatment methods, the methods of surgically eliminating cancerous tissue are disadvantageous in that, since a region around a lesion at which cancerous tissue is located must be excised, the region to be excised is very wide, so that the surgery itself is very difficult, and much time is required for a region operated on to recover completely, and, in addition, a large scar remains on the region after recovery.

In particular, cancerous tissue or the like frequently recurs, and, when this happens, the region around the previously excised lesion must be excised again, thus not only inflicting pain because of the surgery putting the patient at high risk, but also imposing an economic burden.

Accordingly, recently, methods of eliminating cancerous tissue without performing surgery, that is, methods such as transarterial chemoembolization, percutaneous ethanol injection (PEI), systemic chemotherapy, and local thermal therapy, have been widely performed. Among these methods, local thermal therapy is the most effective method of the methods which are widely performed.

The above-described local thermal therapy includes radiofrequency ablation (RFA), microwave ablation, laser ablation, etc. Among these methods, RFA is the most effective method. Such RFA is a method of ablating and destroying only cancerous tissue using RF heat without excising the cancerous tissue when it occurs in a bodily organ, for example, the liver.

In accordance with an embodiment, an apparatus for removing cancerous tissue (cells) by means of RFA as described above may include an RF generator for radiofrequency ablation which generates a radio frequency of a predetermined level, and a single electrode to which the radio frequency generated by the RF generator for RFA is applied.

Meanwhile, the ‘multi-RF generator for radiofrequency ablation’ disclosed in Korean Patent No. 10-739002, which was filed and registered, proposes a technology which not only can extend the range of ablation, but also can improve on safety while efficiently destroying a large-sized cancer tumor by providing radio frequencies from a single RF generator for RFA to a plurality of electrodes, and which enables RFA to be simultaneously performed on two lesions by controlling one or more channels.

The above-described conventional RF generator for RFA monitors variations in the temperature (20° C.˜100° C.) of biological tissue using a thermocouple which is a temperature sensor included at the end of the electrode at the time of performing cauterization (or ablation).

When the temperature of the biological tissue detected by the thermocouple approaches a preset temperature, RF output applied by an RF signal generation unit to the electrode decreases under the control of a data analysis and control unit. Further, the data analysis and control unit calculates output impedance (Z=V/I) using RF voltage and current values detected by a voltage/current detection unit, and increases or decreases the RF output applied by the RF signal generation unit to the electrode according to a change in the output impedance (50Ω˜200Ω).

However, since the above-described conventional technology controls RF output using only the absolute value of output impedance, it is difficult to detect the size and progression of cauterization according to a change in the fine capacitive reactance value of biological cells and then control the RF output.

That is, it is difficult to perform impedance matching due to the characteristics of the capacitive load of the biological cells at the time of performing cauterization, thus making it difficult to perform efficient cauterization.

Further, due to the carbonization of an electrode, impedance easily increases and current decreases, so that it is not easy to realize cauterization to a desired size within a limited time.

Furthermore, since the amount of information required for the overall progression of cauterization is insufficient at the time of controlling RF output according to the temperature, voltage and current, as described above, it is difficult to accurately detect the size of cauterization, thus resulting in the inconvenience of simultaneously observing images of a region to be operated on via fluoroscopy using a diagnostic ultrasound system or the like.

### SUMMARY OF THE INVENTION

Accordingly, the present invention has been made keeping in mind the above problems occurring in the prior art, and an object of the present invention is to provide a method and system for controlling RF output according to the change in impedance of biological cells, which can compare and analyze the phases of RF voltage and current, together with temperature, voltage and current, quantitatively calculate the change in impedance of biological cells, and control RF output based on the change in impedance at the time of performing cauterization.

In order to accomplish the above object, the present invention provides a method of controlling radio frequency (RF) output according to a change in impedance of biological cells, the method controlling RF output applied to an electrode at a time of performing cauterization, comprising a first step of detecting phases of RF voltage and current applied to the electrode and obtaining a difference between the phases; a second step of obtaining a change in impedance such as a resistance component and a capacitive reactance component (XC) of the biological cells at the time of performing cauterization; and a third step of connecting an inductive reactance component (XL) to an RF output terminal so as to cancel the capacitive reactance component (XC) (XC=XL).

Preferably, the inductive reactance component (XL) includes a plurality of inductive reactance components having different values or a variable inductive reactance (XL) having a value varying with external input, and values of the plurality of inductive reactance components or the variable inductive reactance component (XL) are selected or varied according to the capacitive reactance component (XC), and are connected to the RF output terminal.

Preferably, the inductive reactance component (XL) is selected or varied so that it has a value identical to that of the capacitive reactance component (XC), and thus impedance of the biological cells has only a resistance component.

Further, the present invention provides a system for controlling radio frequency (RF) output according to a change in impedance of biological cells, the system controlling RF output applied to an electrode at a time of performing cauterization, comprising a voltage/current phase detection unit for detecting phases of RF voltage and current applied to the electrode; a data analysis and control unit for comparing the phases of the voltage and current detected by the voltage/current phase detection unit with each other, obtaining a difference between the phases, and obtaining a change in impedance such as a resistance component and a capacitive reactance component (XC) of the biological cells at the time of performing ablation; and an inductive reactance selection unit for connecting an inductive reactance component (XL) to an RF output terminal so as to cancel the capacitive reactance component (XC) (XC=XL) under control of the data analysis and control unit.

Preferably, the inductive reactance selection unit includes a plurality of inductive reactance components having different values or a variable inductive reactance component (XL) having a value varying with external input, and the values of the plurality of inductive reactance components or the variable inductive reactance component (XL) are selected or varied according to a value of the capacitive reactance component (XC) and are then connected to the RF output terminal under control of the data analysis and control unit.

Preferably, the inductive reactance selection unit is configured to allow inductive reactance component (XL) thereof to be selected or varied so that the inductive reactance component (XL) has a value identical to that of the capacitive reactance component (XC) under control of the data analysis and control unit, thus enabling impedance of the biological cells to have only a resistance component.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a diagram showing a system for controlling RF output according to the change in impedance of biological cells according to the present invention;

FIG. 2 is a circuit diagram showing an electrical equivalent circuit of the tissue of biological cells; and

FIG. 3 is a graph showing the difference between the phases of RF voltage and current.

### DESCRIPTION OF THE PREFERRED EMBODIMENTS

Hereinafter, embodiments of the present invention will be described in detail with reference to the attached drawings.

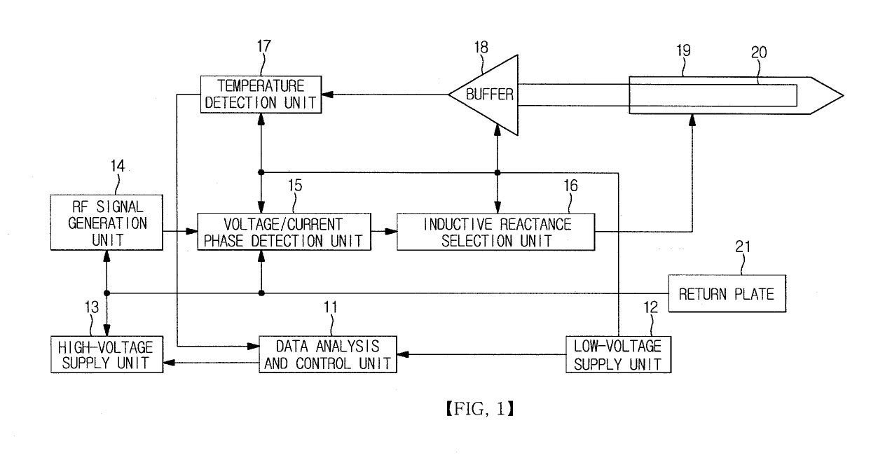

FIG. 1 is a diagram showing a system for controlling RF output according to the change in impedance of biological cells according to the present invention. The system obtains RF output required for cauterization using a high-voltage supply unit 13 for supplying a high voltage to obtain RF output required for cauterization, an RF signal generation unit 14 for receiving the high voltage from the high-voltage supply unit 13, amplifying an oscillation frequency provided by an oscillator (not shown) or the like under the control of a data analysis and control unit 11, and providing the amplified oscillation frequency to an electrode 19 as RF output, a return plate 21, etc.

Further, a temperature value detected by a temperature detection means composed of a thermocouple 20 disposed in the electrode 19, a buffer 18 and a temperature detection unit 17, and the phases of voltage and current detected by a voltage/current phase detection unit 15 are supplied to the data analysis and control unit 11. Accordingly, the data analysis and control unit 11 can compare the phases of the voltage and current detected by the voltage/current phase detection unit 15 with each other, obtain a difference between the phases, and calculate a change in impedance such as the resistance component and the capacitive reactance component (XC) of biological cells at the time of performing cauterization on the basis of the phase difference.

Further, an inductive reactance selection unit 16 connects an inductive reactance component (XL) to an RF output terminal so as to cancel the capacitive reactance component (XC) (XC=XL) under the control of the data analysis and control unit 11.

In this case, the inductive reactance selection unit 16 includes a plurality of inductive reactance components having different values or a variable inductive reactance component (XL) having a value varying with external input. The values of the plurality of inductive reactance components or the variable inductive reactance component (XL) are selected or varied according to the value of the capacitive reactance (XC) and are then connected to the RF output terminal under the control of the data analysis and control unit 11.

That is, the inductive reactance selection unit 16 allows the inductive reactance component (XL) thereof to be selected or varied under the control of the data analysis and control unit 11 so that the inductive reactance component (XL) has a value identical to that of the capacitive reactance component (XC), thereby allowing the impedance of the biological cells to have only a resistance component.

A low-voltage supply unit 12, which is not described in FIG. 1, is a voltage supply unit for supplying low-voltage drive power to the data analysis and control unit 11, the voltage/current phase detection unit 15, the inductive reactance selection unit 16, and the temperature detection unit 17.

Hereinafter, the operation of the present invention will be described in detail.

Similarly to a conventional RF generator for radio frequency ablation (RFA), the system of the present invention is configured such that the RF signal generation unit 14 provided with the high voltage by the high-voltage supply unit 13 amplifies RF output required for cauterization on the basis of oscillation frequency and outputs the amplified RF output to the electrode 19 under the control of the data analysis and control unit 11.

While the RF output is applied using the electrode 19 to a cauterization region, the data analysis and control unit 11 checks the temperature of the cauterization region detected by the temperature detection means including the thermocouple 19, the buffer 18 and the temperature detection unit 17, and then controls the RF output so as to maintain the temperature at the temperature value which can be optionally set.

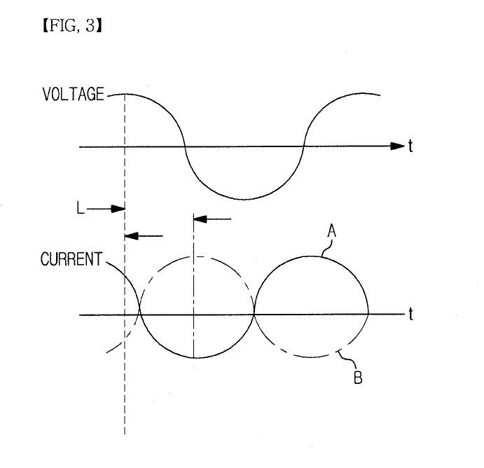

Further, the data analysis and control unit 11 compares the phases of the voltage and current detected by the voltage/current phase detection unit 15 with each other, and detects a difference between the phases, that is, information indicating whether the phase of the voltage is detected to lead the phase of current B, as shown in FIG. 3, or whether the phase of the voltage is detected to lag behind the phase of current A.

That is, the data analysis and control unit 11 obtains a change in the impedance of the biological cells, such as the resistance component and the capacitive reactance component (XC), at the time of performing cauterization.

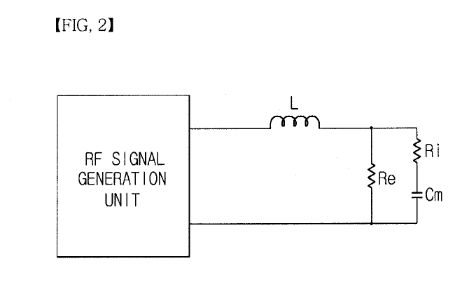

The electrical equivalent circuit of the tissue of the biological cells includes an external biological resistor Re, an internal biological resistor Ri, and the capacitor Cm of a cell membrane, as shown in FIG. 2. In this case, the internal biological resistor Ri and the cell membrane capacitor Cm, connected in series with each other, are connected in parallel with the external biological resistor Re.

The capacitive reactance component (XC) of the cell membrane capacitor Cm is given by the following Equation (1).

$$XC = \frac{1}{2 π fCm} \qquad ( 1 )$$

Next, the inductive reactance component (XL) of the inductor L for canceling the capacitive reactance component (XC) is given by the following Equation (2).

XL=2πfL (2)

Therefore, the output impedance Zt is given by the following Equation (3).

$$\begin{matrix}Zt = Re // Ri ( 1 / 2 π fCm ) \\ = Re \times ( Ri + 1 / 2 fCm ) / Re + Ri + ( 1 / 2 π fCm )\end{matrix} \qquad ( 3 )$$

The data analysis and control unit 11 which has obtained the capacitive reactance component (XC) together with the resistance component, as in the above description, drives the inductive reactance selection unit 16 and enables the inductive reactance component (XL) to be connected to the output terminal of the RF signal generation unit 14 so as to cancel the capacitive reactance component (XC) (XC=XL).

Therefore, since the inductive reactance component (XL) of the inductive reactance selection unit 16 and the capacitive reactance component (XC) of the biological cells have the same value by the driving of the inductive reactance selection unit 16, only a resistance component remains as the impedance of the biological cells.

In this case, the inductive reactance selection unit 16 may include a plurality of inductive reactance components having different values or a variable inductive reactance component (XL) having a value varying with external input. The values of the plurality of inductive reactance components or the variable inductive reactance component (XL) are selected or are varied under the control of the data analysis and control unit 11 which checks the value of the inductive reactance component (XL).

Therefore, the present invention is advantageous in that, the phases of RF voltage and current, together with temperature, voltage and current, are compared and analyzed, so that the change in impedance of biological cells can be quantitatively calculated, and RF output obtained at the time of performing cauterization can be controlled based on the change in impedance, thus improving cauterization efficiency at the time of performing cauterization.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A method of controlling radio frequency (RF) output according to a change in impedance of biological cells, the method controlling RF output applied to an electrode at a time of performing cauterization, comprising:
a first step of detecting phases of RF voltage and current applied to the electrode and obtaining a difference between the phases;
a second step of obtaining a change in impedance such as a resistance component and a capacitive reactance component (XC) of the biological cells at the time of performing cauterization; and
a third step of connecting an inductive reactance component (XL) to an RF output terminal so as to cancel the capacitive reactance component (XC) (XC=XL).

2. The method according to claim 1, wherein the inductive reactance component (XL) includes a plurality of inductive reactance components having different values or a variable inductive reactance (XL) having a value varying with external input, and values of the plurality of inductive reactance components or the variable inductive reactance component (XL) are selected or varied according to the capacitive reactance component (XC), and are connected to the RF output terminal.

3. The method according to claim 1 or 2, wherein the inductive reactance component (XL) is selected or varied so that it has a value identical to that of the capacitive reactance component (XC), and thus impedance of the biological cells has only a resistance component.

4. A system for controlling radio frequency (RF) output according to a change in impedance of biological cells, the system controlling RF output applied to an electrode at a time of performing cauterization, comprising:
a voltage/current phase detection unit for detecting phases of RF voltage and current applied to the electrode;
a data analysis and control unit for comparing the phases of the voltage and current detected by the voltage/current phase detection unit with each other, obtaining a difference between the phases, and obtaining a change in impedance such as a resistance component and a capacitive reactance component (XC) of the biological cells at the time of performing ablation; and
an inductive reactance selection unit for connecting an inductive reactance component (XL) to an RF output terminal so as to cancel the capacitive reactance component (XC) (XC=XL) under control of the data analysis and control unit.

5. The system according to claim 4, wherein the inductive reactance selection unit includes a plurality of inductive reactance components having different values or a variable inductive reactance component (XL) having a value varying with external input, and the values of the plurality of inductive reactance components or the variable inductive reactance component (XL) are selected or varied according to a value of the capacitive reactance component (XC) and are then connected to the RF output terminal under control of the data analysis and control unit.

6. The system according to claim 1 or 2, wherein the inductive reactance selection unit is configured to allow inductive reactance component (XL) thereof to be selected or varied so that the inductive reactance component (XL) has a value identical to that of the capacitive reactance component (XC) under control of the data analysis and control unit, thus enabling impedance of the biological cells to have only a resistance component.
